(19) United States
(12) Patent Application Publication
(10) Pub. No.: US 2014/0243770 A1
(43) Pub. Date: Aug. 28, 2014

(54) SURGICAL SPONGE HAVING RADIOPAQUE ELEMENT AND METHOD OF MANUFACTURE

(71) Applicant: Brian E. Stewart, Newport Coast, CA (US)

(72) Inventor: Brian E. Stewart, Newport Coast, CA (US)

(73) Assignee: Patient Safety Technologies, Inc., Irvine, CA (US)

(21) Appl. No.: 13/779,467

(22) Filed: Feb. 27, 2013

(51) Int. Cl. A61F 13/44

(52) U.S. Cl. CPC A61F 13/44
USPC 604/362; 112/475.01

(57) ABSTRACT

A surgical sponge comprises a sheet of absorbent material having at least one elongated radiopaque element heat sealed thereto. The sponge is folded and sewn in particular ways to assure that the radiopaque length of material remains firmly secured to the sponge with enhanced visibility under fluoroscopic imaging.

### BACKGROUND OF THE INVENTION

1. Field of the Invention

The present invention relates generally to medical devices and methods. More particularly, the present invention relates to surgical sponges having radiopaque elements and methods for their manufacture.

Surgical sponges are used during surgical procedures to absorb blood and other body fluids in and around a surgical site. Unlike ordinary household sponges, surgical sponges are typically formed from a woven cotton or other mesh similar to surgical gauze.

It is critical that all surgical sponges used in a procedure be removed from the patient at the end of the surgery and prior to closure of the surgical site. Great efforts are made to make sure that no surgical sponges are inadvertently left inside patients after surgical procedures, and recent advances in counting sponges using barcode technologies and other methods of incorporating machine readable information have shown great promise.

Even though such scanning techniques promise to greatly reduce or even eliminate the risk of leaving surgical sponges in the patient, it is still desirable to incorporate radiopaque materials in a surgical sponge to make them more readily apparent using x-rays. For example, when the sponge counting methods indicate that the sponge is missing and likely in a patient, it can be difficult to locate the sponge, and use of x-ray fluoroscopy can be of great benefit if the sponge has radiopaque elements incorporated in it to confirm the sponge is not inside the patient. Moreover, it is always useful to have a backup procedure for locating lost surgical sponges if for any reason other techniques fail.

A variety of radiopaque materials and elements have been incorporated into surgical sponges. One particularly useful radiopaque element comprises a thread or element composed of a radiopaque material or impregnated with a radiopaque material. The thread or filament can be woven into the woven sponge material thus assuring that it remain in place. The need to weave the thread or filament into the sponge, however, can make fabrication more difficult and costly. Additionally, the thread or filament can be attached to the surface of the sponge material utilizing one or more adhesion methodologies. The use of adhesion methodologies can make fabrication more difficult and costly and further introduces the risk the thread or filament may not stay attached to the sponge surface.

For these reasons, it would be desirable to provide alternative and improved surgical sponge structures and methods for their manufacture. In particular, it would be desirable to provide alternative methods for incorporating elongated radiopaque elements, such as threads, filaments, yarns, and the like, into a surgical sponge structure with maximum efficiency, reduced likelihood of the radiopaque element becoming dislodged from the sponge, and minimum cost. The resulting surgical sponge structure should be amenable and useful for all sizes of surgical sponges, including even the large sponges referred to as “lap pads” or “abdominal pads,” and should further be compatible with folding such sponges into smaller configurations. At least some of these objectives will be met by the inventions described below.

2. Description of the Background Art

Surgical sponges and methods for their manufacture are described in U.S. Pat. Nos. 4,244,369; 4,626,251; 4,704,109; and 5,112,325; and in U.S. Patent Publ. No. 2005/0016776.

### SUMMARY OF THE INVENTION

The present invention provides improved surgical sponge structures and methods for their manufacture. The sponge structures have at least one elongated radiopaque element adhered to a surface of a single layer of absorbent material. The elongated radiopaque element can be any long, narrow element capable of being adhered to a surface of the absorbent material, typically being an extruded elongated cylinder having a diameter in the range from 0.1 to 1.5 mm, typically being about 0.7 mm. In exemplary embodiments, the radiopaque element can be a “thread” which softens or partially melts to adhere to the absorbent sheet, typically being composed at least partially of a heat-meltable polymer, such as a polyvinylchloride (PVC). Usually, the heat-meltable polymer will be impregnated with a radiopaque material, such as barium sulfate (BaSO₄).

This sheet having the elongated radiopaque element thereon is folded at least once so that the elongated radiopaque element is captured between two folds, and the elongated radiopaque element maybe “ironed” or otherwise heat sealed to the surface of at least one of the folds. In the exemplary embodiments, the thread is heat sealed to an inner surface of the sheet prior to folding. In other embodiments, the thread could be ironed or otherwise heat sealed between the sheets after folding.

In addition to such heat sealing, the elongated radiopaque element is further secured to the folded absorbent material by sewing. Typically, the folded material will be sewn along each edge (four in the case of square and rectangular surgical sponges) and optionally at least once across a central portion of the sheet. In this way, the elongated radiopaque element will be sewn over at at least three places where the sew lines cross the element. Sewing the surgical sponge above the edges further assures the dimensional stability of the sponge and adds a further degree of containment for the radiopaque element. That is, should the radiopaque element somehow become dislodged from both the heat sealing and the sewing, the fact that the edges of the sponge are sewn will in all likelihood contain the element within the sponge. In many cases, the sponge will be further folded after sewing and incorporation of the elongated radiopaque element. For example, the sponge may be folded once along a centerline in one direction and then folded a second time along an orthogonal centerline. In further exemplary embodiments, the sponge may further include a radiopaque tab or label at one corner to provide an alternative radiopaque artifact when observed under fluoroscopic imaging. Such surgical sponges may further include a loop, typically as part of the radiopaque label or tab, in order to help retrieve the sponge.

In a first specific aspect of the present invention, a surgical sponge comprises a sheet of absorbent material, typically a gauze sheet, and at least one radiopaque length of material. Two halves of the sheet are folded along a center fold line, and at least one radiopaque length of material is disposed between the halves along a line parallel to the fold line. All edges of the folded sheet are sewn together, and a center portion of the folded sheet is may optionally further sewn thereacross. In this way, at least two of the edge sew lines and the optionally a third center sew line will cross the radiopaque length of material, assuring that the radiopaque length of material is captured between the folded halves of the sheet.

In specific embodiments, the length of radiopaque material comprises a thread impregnated with barium sulfate or other radiopaque filler or material. The thread may comprise virtually any structure, such as a filament, a yarn, a ribbon, and the like, but will most typically comprise a filament extruded from a heat-meltable polymer, such as polyvinylchloride. In such cases, the polymer thread will typically have been heated to adhere the thread to the sheet of absorbent material, usually prior to folding, in order to further assure that the radiopaque length of material will not be lost from the sheet when it is in use in a patient.

In further specific embodiments, the free edges of the sheet (other than the edge folded along the center fold line), will be folded under before sewing. Such folding of the edges provides a stronger base or matrix to receive the sewing and reduces the risk of the edges fraying during use.

In a second specific aspect of the present invention, a method for fabricating a surgical sponge comprises providing a rolled sheet of an absorbent material, typically surgical gauze, having a width and an axial centerline. The sheet is continuously drawn from the roll, and a length of radiopaque material is continuously fed over an inner surface of the sheet along the lines based laterally outwardly from the centerline. The sheet is folded along the centerline to entrap the radiopaque length of material between the inner surfaces of the folded halves of the sheet. The folded sheet is cut laterally multiple times to form a plurality of separate sponges. Each separate sponge is sewn along each edge and optionally may be sewn along an additional line between the edges so that the radiopaque length of material is crossed by at least two sew lines and optionally three sew lines. In this way, the radiopaque length of material is firmly held between the folded layers of the absorbent sheet of material.

In specific embodiments, the method further comprises sealing the length of radiopaque material to the surface of at least one of the absorbent material halves. More specifically, the radiopaque length of material comprises a thread composed of a polymeric material, and sealing comprises heating the thread to melt and adhere the polymer to the sheet. Still more specifically, heating may comprise ironing the sheet to at least partially melt the polymeric material prior to folding and cause the thread or other length of material to adhere to the inner surface of the sheet of absorbent material before folding.

In further specific embodiments, the methods of the present invention may further comprise attaching a radiopaque tab to one corner of the sewing sponge. At least one edge of the folded sponge other than the edge folded along the centerline will be folded under prior to sewing. The sewing sponge may be further folded one or more times, often being folded two or more times.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a schematic illustration of a system configured to produce a folded surgical sponge structure in accordance with the principles of the present invention.

FIG. 1A illustrates an alternative embodiment of the system of FIG. 1 where a single heated roller us used to adhere a radiopaque element to a sheet of absorbent material.

FIG. 2 is a cross-sectional view taken along line 2-2 of FIG. 1.

FIG. 3 is a cross-sectional view taken along line 3-3 of FIG. 1.

FIG. 4 is a cross-sectional view taken along line 4-4 of FIG. 1.

FIG. 5 illustrates cutting of the folded surgical sponge structure produced as shown in FIG. 1 into a plurality of individual surgical sponges.

FIG. 6 illustrates a folded, cut and sewn surgical sponge as can be produced by the system of FIGS. 1 and 5.

FIG. 7 is a detailed, edge view taken along line 7-7 of FIG. 6.

FIG. 8 is a detailed, edge view taken along line 8-8 of FIG. 6.

FIG. 9 illustrates the surgical sponge of FIG. 6 folded along a centerline transverse to a radiopaque element.

FIG. 10 illustrates the folded sponge of FIG. 9 further folded along a line parallel to the radiopaque element.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

### DETAILED DESCRIPTION OF THE INVENTION

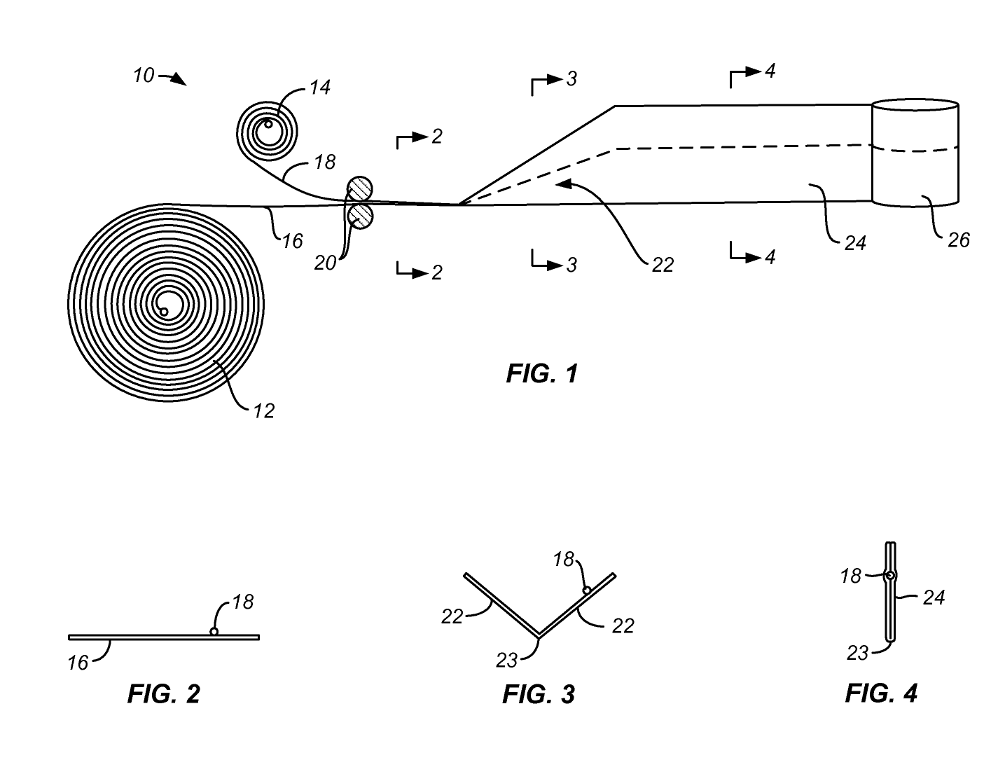

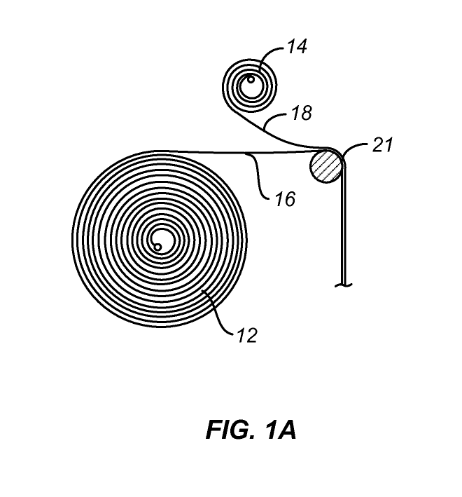

Referring to FIG. 1, a system 10 for producing folded surgical sponges in accordance with the principles of the present invention includes a roll 12 or other source of absorbent material, typically a cotton gauze, in the form of a single layer. Alternatively, the absorbent material could be provided in two, three, or more layers. The system 10 further includes a coil of radiopaque material, typically a barium sulfate-impregnated polyvinylchloride thread. A layer of absorbent material 16 from the roll 12 and length of the radiopaque material 18 from the coil 14 are brought together and heat sealed as they pass between heated rollers 20, as shown in both FIG. 1 and FIG. 2. Alternatively, the radiopaque material 18 from the coil 14 and layer of absorbent material 16 may be brought together and heat sealed as they pass over a single heated roller 21, as shown in FIG. 1A.

The sheet of material 16 having the adhered elongated length of radiopaque material 18 is then folded into halves 22 along a centerline 23, as shown in FIGS. 1 and 3, until the sheet is fully folded, as shown in FIGS. 1 and 4. Typically, a blade or other folding structure is used to produce the fold, but such conventional structure is not shown in figures for simplicity of presentation. The folded sheet 24 of FIG. 4 is then rolled onto a takeup roll 26 and transported for further processing.

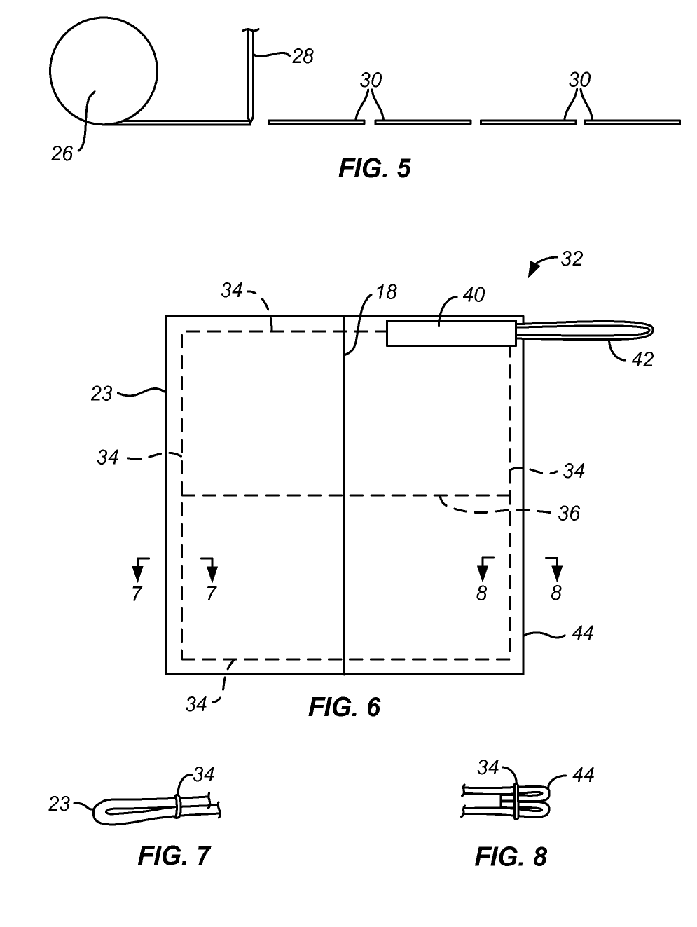

As shown in FIG. 5, the roll of folded absorbent sheet material 26 is then unrolled and a blade 28 used to cut the material into a plurality of individual cut sponge structures 30. It will be appreciated that in other systems, the intermediate steps of rolling and unrolling the folded sheet material could be eliminated.

The individual cut sponge structures 30 are then sewn along each edge and also along a centerline, as shown in FIG. 6. A sewn and completed surgical sponge 32 is sewn along edge sew lines 34 as well as along a center sew line 36, where at least two of the edge sew lines 34 and the center sew line 36 cross over the radiopaque length of material 18. Sewing the radiopaque length of material 18 at at least three locations ensures that the radiopaque material will remain entrapped within the folded sides 22 of the sponge. Heat sealing of the radiopaque thread to the sheet further ensures that the radiopaque element 18 will not be lost.

The folded centerline ridge 23 is sewn without further folding or modification, as shown in FIG. 7. The unfolded edges 44 of the sponge 32, however, are typically folded under as shown in FIG. 8 prior to sewing. Such folding under provides for a stronger sew line (with more material to provide purchase or grasp of the sew line 34) and also helps prevent unraveling or fraying of the edge.

In the specific embodiments, a radiopaque tab or ribbon 40 may be sewn into one corner of the surgical sponge 32. The tab will typically further include a loop or string 42 which is useful for retrieving and storing the sponges.

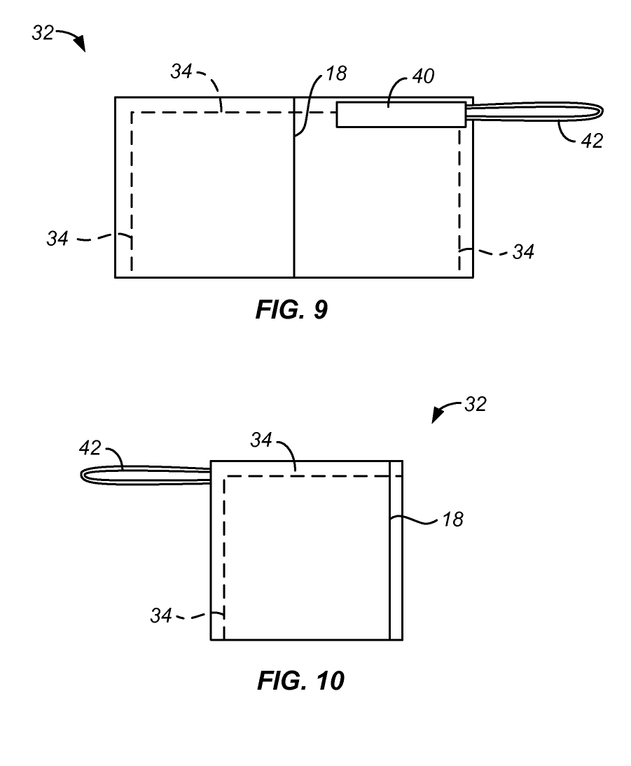

As shown in FIG. 9, the folded surgical sponge of FIG. 6 can be further folded along a centerline in the first direction. The sponge may be folded at least a second time along an orthogonal fold line, as shown in FIG. 10. The sponges 32 may then be used in either the full folded configurations or may be unfolded partially or fully prior to use.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A surgical sponge comprising
a sheet of absorbent material; and
at least one radiopaque length of material;
wherein two halves of the sheet are folded over along a center fold line and the at least one radiopaque length of material is disposed between the halves along a line parallel to the fold line; and
wherein all edges of the folded sheet are sewn together and a center portion is sewn across so that the radiopaque length of material is sewn to the absorbent material at at least three locations.

2. A surgical sponge as in claim 1, wherein the folded halves of the sheet are sewn together across at least one center portion so that the radiopaque length of material is sewn to the absorbent material at least three locations.

3. A surgical sponge as in claim 1, wherein the length of material comprises a thread impregnated with barium sulfate.

4. A surgical sponge as in claim 3, wherein the thread comprises a polymer.

5. A surgical sponge as in claim 4, wherein the polymer thread has been heated to adhere the thread to a surface of the sheet of absorbent material.

6. A surgical sponge as in claim 1, wherein the edges of the spine, except for the edge along the center fold line, are folded under before sewing.

7. A surgical sponge as in claim 1, further comprising a radiopaque strip attached to one corner of the folded, sewn sponge.

8. A method for fabricating a surgical sponge, said method comprising:
providing a rolled sheet of absorbent material having a width and an axial centerline;
continuously drawing the sheet from the roll;
continuously feeding a length of radiopaque material over an inner surface of the sheet of along a line spaced laterally outwardly from the centerline.
folding the sheet along the centerline to trap the radiopaque length of material between the inner surfaces of folded halves of the sheet;
cutting the folded sheet laterally to form a plurality of separate sponges; and
sewing each separate sponge along each edge such that the radiopaque length of material is firmly held between the folded halves of the absorbent material.

9. A method as in 8, further comprising sewing each separate sponge additionally between the edges along a line that intersects the radiopaque length of material

10. A method as in claim 8, further comprising sealing the length of radiopaque material to at least one of the absorbent material halves.

11. A method as in claim 10, wherein the length of material comprises a polymeric thread and wherein sealing comprises heating the thread to adhere the polymer to the sheet.

12. A method as in claim 11, wherein heating comprises ironing the length of radiopaque material onto the inner surface of the sheet of absorbent material before folding.

13. A method as in claim 8, further comprising attaching a radiopaque tab, to one corner of the sewn sponge.

14. A method as in claim 8, wherein at least one of the edges of the folded sponge other than the edge along the centerline are folded under prior to sewing.

15. A method as in claim 14, wherein the sewn sponge is folded at least one more time.

16. A method as in claim 14, wherein the sewn sponge is folded at least two more times.
